(19) United States
(12) Patent Application Publication
(10) Pub. No.: US 2008/0255707 A1
(43) Pub. Date: Oct. 16, 2008

(54) Method and system for categorizing blood glucose tests at test time in a portable device or later in a downloading program and then analyzing the categories separately

(76) Inventors: Harry R. Hebblewhite, Atlanta, GA (US); Paul C. Davidson, Atlanta, GA (US)

Correspondence Address: Harry R Hebblewhite, Paul C Davidson;Atlanta Diabetes Associates, Suite 2080, 77 Collier Rd, NW, Atlanta, GA, 30309, US

(21) Appl. No.: 11/784,807

(22) Filed: Apr. 10, 2007

(51) Int. Cl. G05D 7/00; A61B 5/145; A61K 9/22; A61M 5/142

(52) U.S. Cl. 700/283; 600/347; 604/504; 604/892.1; 700/282; 702/19

(57) ABSTRACT

A four-part method for analyzing data of a patient's blood glucose (BG) tests or other parameters e.g. carbohydrate intakes and discrete insulin doses (boluses): 1. An LCD-button on the portable glucose meter or insulin pump, that allows flagging or assigning a category to each blood glucose test at test-time. Examples of categories: pre-meal BG tests, post-meal BG tests. 2. In the computer-based downloading program, features for changing the boundaries of the meal-oriented time-intervals by the faster methods of: a) mouse-dragging the time-boundary lines on a scatter chart, b) placing stretchable rectangles around the data points of interest. 3. Features in the computer-based downloading program, for digitally identifying the time of the meal and digitally assigning the category to the pre-meal BG tests and to the post-meal BG tests. 4. Features in the computer-based downloading program, for statistically analyzing and graphically-displaying the differently-categorized data separately or together in combinations.

### CROSS REFERENCE TO RELATED APPLICATION

Method and System for Categorizing Blood Glucose Tests at Test Time in a Portable Device and then Analyzing the Categories Separately in a Downloading Program; Inventors: Paul C. Davidson; Harry R. Hebblewhite, Postcard Receipt 113260 US PTO 60/790,741 040906

### FIELD OF THE INVENTION

The invention relates generally to the field of digital aids to assist in the treatment of diabetic patients who use insulin pumps, blood glucose meters, or other portable devices.

### BACKGROUND OF THE INVENTION

Diabetes Mellitus has been treated for many years by insulin injection. The invention of the portable blood glucose meter has made a large improvement in diabetes care. This was followed by PC-based software to download the Blood Glucose test results (BG's) from the memories of the meters, graphically display them, analyze them, and print reports suitable for the patient's chart folder. Additionally, there have been three recent advances in insulin-delivery systems: The Insulin Pump, new insulin formulas for Multiple Dose Injection, and Inhaled Insulin. The delivery systems are discussed first below:

### Developments in Insulin Delivery Systems:

THE INSULIN PUMP: The invention of the insulin pump revolutionized diabetes care. It is a battery-powered device about the size of a pager. It contains a cartridge of insulin and pumps the insulin through a flexible tube into the patient via an “infusion set”, which is a small plastic needle or “canula” fitted with an adhesive patch. The invention of the pump makes it possible to adopt a typical insulin regimen as follows: basal insulin is injected slowly and continuously at a rate that can be programmed to change multiple times during the day (about 4 or 5 changes per day is common). Additionally, boluses of insulin can be injected on command by the patient. There are two main types of boluses:
Meal Boluses are infused just before a meal in an amount, which is proportional to the glycemic effect of the meal. This is generally proportional to the number of grams of carbohydrate in the meal. The proportionality constant is a personalized number called the Carbohydrate-to-Insulin Ratio, CIR. It is used as follows:

Meal Insulin Bolus=(grams of carbohydrates in the meal)/CIR

This calculation is generally performed by the patient, but there are pump models that can store the patient's CIR in memory and require only the grams of carbohydrate in the meal as the input.
Correction Boluses are infused immediately after a Blood Glucose test (BG) has been performed; the amount of the correction bolus is proportional to the error of the blood glucose concentration from the patient's personalized Target Blood Glucose. The proportionality constant is a personalized number called the Correction Factor, CF. It is used as follows:

Corrective Insulin Bolus=(BG−Target)/CF

Sometimes the Meal Bolus and Correction Bolus are combined in the software of the portable device to give a Combination Bolus.

There are two main types of Blood Glucose test, each with a different Target:
Pre-meal BG's: Administered in a fasting state, just before a meal. Pre-meal BG's are very common.
Post-meal BG's: Administered about two hours after a meal. Corrective Boluses are sometimes taken. Some patients use these tests for information only and do not bolus. Post-meal BG's are less common.

Some brands of insulin pump have a feature that uses an experimentally-determined formula for the curve of remaining insulin versus time after a previous bolus. This will be referred-to hereinafter as Insulin Remaining. This is used to apply a deduction to a newly-ordered bolus to prevent overdosing. The formula for the new bolus is:

[New bolus delivered]=[Calculated Meal Bolus+Calculated Correction Bolus]−[Deduction for Insulin Remaining]. (1)

MULTIPLE DOSE INJECTION (MDI): Advances are being made in developing different types of insulin. Some are very long acting and non-peaking. The long-acting insulin can be injected as infrequently as once per day in a regimen very similar to a pump patient's basal insulin regimen. Injections of rapid-acting types of insulin can be given as meal and correction boluses. These insulins are available in portable “pens” (named for their resemblance to writing implements).

INHALED INSULIN: Inhaled insulin delivery systems are under development for short-acting insulin. Inhaled insulin enters the blood very rapidly. Inhaled insulin may be used in place of the short-acting in a system very much like MDI.

### Developments in Blood Glucose Meters and Downloading SOFTWARE:

Much of the blood glucose testing technology is in the test strips, which contain chemicals and electrodes manufactured to a high precision. Their purpose is to convert the blood glucose concentration to an electrical resistance. Within the portable meter body are a battery, chips and digital circuitry that read the resistance and convert the electrical signal to a digital numerical value and store it in digital memory together with the time and date. The meter manufacturers also provide PC-based software to download the memories of the meters and insulin pumps, analyze the data from the latest user-specified calendar period, and display printable reports. The reports typically contain the following:
Trend chart—With blood glucose on the y axis and time in days on the x axis, the chronological trend is shown for the selected calendar period.
Standard Day chart—With blood glucose on the y axis and twenty-four hours of time on the x axis, the data from all the days in the calendar period are superimposed on the same “standard day”.
Logbook table—the standard day is divided into time intervals roughly corresponding to: before breakfast, after breakfast, before lunch, after lunch, before dinner, after dinner, bedtime, and sleep. The programs for some currently-available meters do not have after each meal. All the BG's for the calendar period being analyzed are sorted into the time intervals which are displayed in the left-to-right direction. The BG's are placed chronologically in the downward direction, so that a long calendar period with many BG's will cause several pages of Logbook table.
Logbook statistics table: Calculations such as BG count, average, standard deviation, count of highs, and count of lows, are calculated for each of the time intervals in the typical day shown in the Logbook. Each of these calculations includes all the tests over the calendar period that fall into the given time interval in the typical day. The display format may be very similar to the logbook, even to the extent of placing the average numbers as the first or last line in the logbook and distinguishing it with boldface, shading, or some other means.

Typically, the time-boundaries for the time intervals in the Logbook table are adjustable by the user. The positioning of these boundaries greatly affects the final results. The intention of the manufacturers was for the pre-meal time interval boundaries to be placed around the pre-meal BG's and for the post-meal time interval boundaries to be placed around the post-meal BG's. This is easy for the user (the clinician) to accomplish if the patient keeps very regular mealtimes. But few patients do. In the present state-of-the-art of meter downloading programs, the time-interval boundaries are moved by first navigating to a different part of the program to a “Patient's Settings” page containing a table of boundary times for the time intervals. The times may be changed by placing the cursor by mouse and typing in the desired time or by a scrolling combination box. These methods are time-consuming.

In the present state of the art the number of time-intervals is fixed. Some downloading programs have five time-intervals (no post-meal intervals), which is appropriate for a typical well-organized person who eats three meals and a bedtime snack, uses no post-meal BG's and takes a late-night BG test. Some downloading programs have eight intervals (including post-meal intervals), which helps somewhat for patients whose schedule is complicated or disorganized. However, the number of intervals cannot be changed. If there are too many intervals, the extras must be hidden by reducing them to the minimum size and placing them in a time region with few or no BG's. If they are hidden, the names of the remaining intervals are often no longer applicable, e.g. the “Pre-Breakfast” interval may be placed in the dark hours and the “Post-Breakfast” interval may take over the job of “Pre-Breakfast”, but the names cannot be changed.

Time-intervals have important uses, but the current system of trying to employ them to differentiate pre-meal BG's from post-meal BG's does not work well. One of the reasons is that there is not a sharp line between pre-meal and post-meal; some early post-meal BG's might fall into the pre-meal interval, or some late post-meal BG's might be so close to the next meal that they get counted as pre-meal BG's.

Pre-meal and post-meal BG's are very different. Consumption of a carbohydrate-containing meal generally causes a roughly bell-shaped or peak-shaped excursion of blood-glucose concentration peaking about an hour after the meal and dissipating by three hours after the meal. The pre-meal tests are near the Target and occupy the flat valleys between the post-meal excursions. They give real-time knowledge to the patient, which is especially important because these are the time periods when the blood glucose is likely to go low, causing hypoglycemia, a condition that can lead to unconsciousness and brain damage. These pre-meal BG's also give information to the clinician about the Basal Insulin of a pump patient or the long-acting dose of a Multiple-Dose-Injection patient. They also give information about the previous Meal Bolus in the sense that errors in the previous Meal Bolus would lead to an off-Target pre-meal test result before the next meal.

The post-meal tests are usually somewhere on the top or sides of the post-meal peak. The variability of the peaks and of the position of the test on the peak means that the post-meal tests have a large standard deviation, and they are usually much higher than the pre-meal tests. They provide real-time knowledge to the patient. They also provide information to the clinician about the accuracy of the patient's Meal Boluses, but this information is subject to the same high variability as the tests themselves.

Because of the different meaning and different uses of the pre-meal and post-meal BG's, the present state-of-the-art of displaying and analyzing them together is inferior. In some downloading systems, the normally-high post-meal BG's are averaged with the pre-meal test results. If these averages are treated as all pre-meal tests, then more insulin is prescribed, raising the risk of hypoglycemia. It would be a great benefit for the clinicians to be able to separate these two types of BG in order to view them separately in scatter charts and analyze them separately. The present invention helps to solve this problem. Another deficiency of the present state-of-the-art downloaders is that Standard Day scatter charts are available only for BG tests. It would be a great help to the clinician if other parameters could be treated in this same way, for instance carbohydrate intake, Meal Boluses, Correction Boluses, Combined Boluses, and Remaining Insulin.

Another deficiency of the present state-of-the-art example of a use of the invention is to differentiate regular BG's from those (usually high) BG's performed after correction of a low BG by the consumption of sugar. This is explained in more detail: When a patient has a low BG, he or she can often feel it as a change in general mood or alertness. This is the cue to take a BG test, or this first test may be routine. Either way, if the meter confirms a low BG of, say 56 mg/dl, then the standard practice is to take glucose tablets. There are rules-of-thumb about how many tablets to take to raise the blood glucose by a certain amount. However, the patient may mis-estimate, or not have any glucose tablets at hand and substitute a soft drink. The result may be an overshoot of the Target on the high side. About this time, the patient will very likely be taking another BG test to see if the low BG has been successfully treated. It is this high BG that is of special interest because it was induced by the sugar, not by the patient's prescribed regimen. These high BG's will appear in the downloading program and the clinician may interpret their cause as an error in the prescribed insulin regimen. Also, they will be included in the averages, making the averages artificially high. The present invention enables the regular BG's to be analyzed by themselves without the error-causing inlusion of the post-sugar-treatment highs.

### BRIEF SUMMARY OF THE INVENTION

The present invention is a digital aid that will improve upon current diabetes care by making it possible for the physician or practitioner to analyze in greater detail the data downloaded from the patient's portable devices (blood glucose meters and insulin pumps). Such data are comprised of the patient's past history of parameters such as: Blood Glucose (BG) tests, Carbohydrate Intakes, Meal Boluses, Correction Boluses, Combined Boluses, and Remaining Insulin deductions from boluses. The discussion below will focus on BG data but the same methods apply to the other parameters mentioned. The invention is in four parts:

1) In the blood glucose meter, insulin pump or other portable device: a physical button or screen selector to enable the patient at test-time to assign to each BG test result one or more category labels. Simple embodiments have one yes-or-no choice whose meaning is known to the patient and clinician. Complex embodiments allow multiple assignments of text-string categories including write-ins and BG's may have several categories simultaneously. Examples of categories: pre-meal BG's, post-meal BG's, and BG's after sugar-treated lows.

When the patient visits the clinician, the clinician downloads the portable device using the computer-based downloading software which has the following features:

2) Features for changing the boundaries of the meal-oriented time-intervals by the new and faster graphical methods of: a) mouse-dragging boundary lines on a Standard Day scatter chart, b) placing stretchable rectangles around the data points of interest on a Standard Day scatter chart. c) enclosing the data of interest in closed freehand mouse-drawn figures on a Standard Day scatter chart. Both b) and c) allow an odd-shaped group of data points to be enclosed together in a time-interval and leave anomalous data points out of the enclosures.

3) Features for digitally determining the meal time within a time-interval, and subsequently to determine pre-meal BG's, and post-meal BG's and digitally assigning the categories (“pre-meal”, and “post-meal”) to these BG's. The post-meal BG's are determined for each meal-containing interval of each day as those falling within a post-meal time window, which are arranged to start at a delay-time after the meal and run until an end-time after the meal.

4) Features allowing data with different categories to be displayed graphically and analyzed together, separately, or in different combinations. Example: Viewing and analyzing the pre-meal BG's by themselves (without the post-meal BG's or the post-sugar-treatment BG's) is a valuable method of analysis made possible by this invention. Viewing and analyzing Meal Boluses, Correction Boluses and Carbohydrate consumption is also made possible. Features for selecting the method of category assignment for display purposes, i.e. in accordance with Part 1), Part 2) or Part 4) of this Brief Description section.

### BRIEF DESCRIPTION OF THE FIGURES

Computer-generated data was used in these simulations:

FIG. 1 illustrates a present state-of-the-art report form from a typical meter of today. It combines the features of the Standard Day report form and the Logbook Statistics report form. No differentiation is made between the two categories of BG's, pre-meal BG's and post-meal BG's.

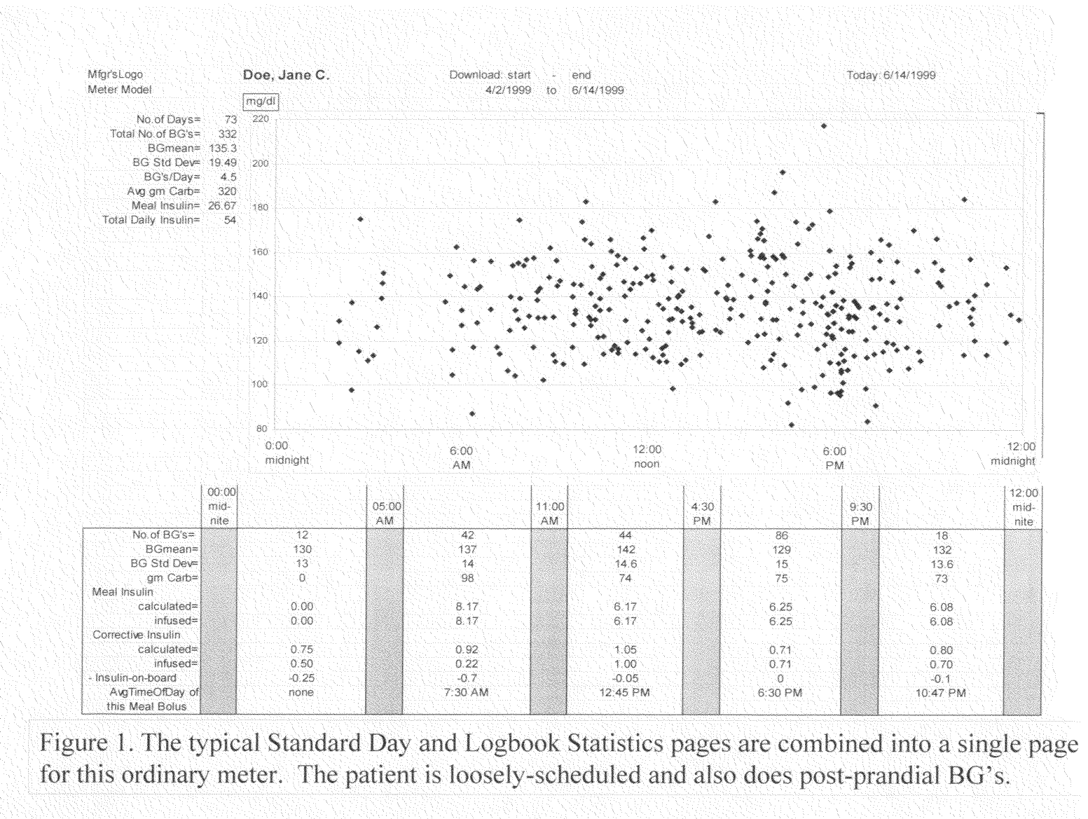

FIG. 2 illustrates an exemplary embodiment in which a single two-valued category parameter was used to differentiate two categories: pre-meal BG's (not flagged) and post-meal BG's (flagged). It shows the combined Standard Day and Logbook Statistics report forms in which the pre-meal BG values are shown as solid characters, and the post-meal BG's are shown as hollow squares. The computer screen version of this form may display the different data in different colors.

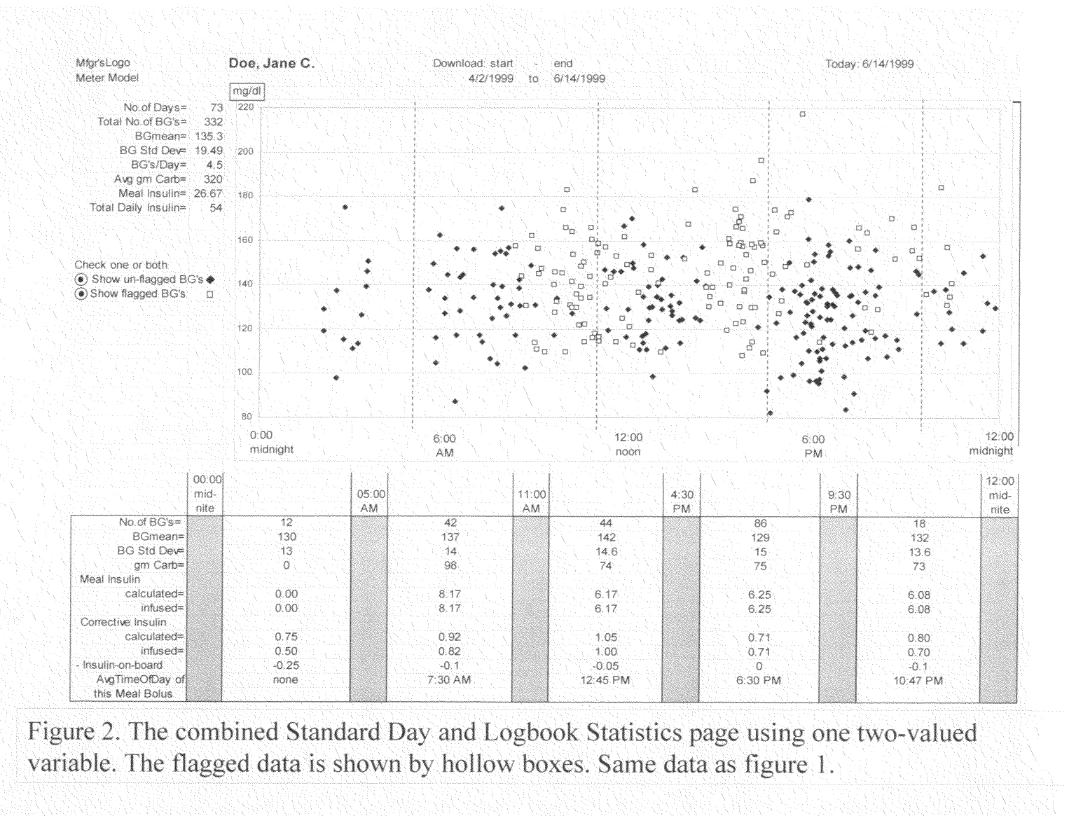

FIG. 3 illustrates an exemplary embodiment in which a single two-valued category parameter was used to differentiate pre-meal BG's from post-meal BG's. It shows the combined Standard Day and Logbook Statistics report form in which only the pre-meal (flagged) BG values are shown. It is very easy for the user to see where the mealtimes are and to properly place the time-interval boundaries. The statistics at the bottom pertain only to the flagged BG's.

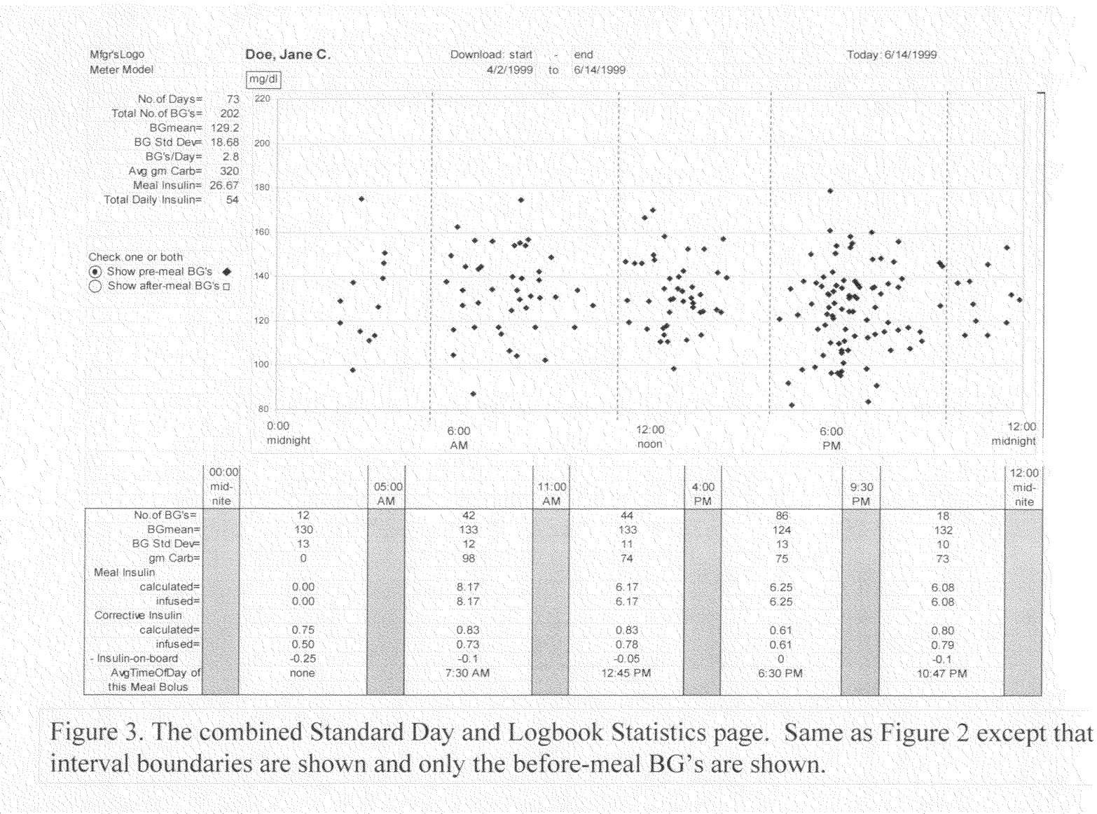

FIG. 4 illustrates an exemplary embodiment in which a single two-valued category parameter was used to differentiate pre-meal BG's from post-meal BG's. It shows the combined Standard Day Report Form in which only the post-meal BG's are shown. The flagged and un-flagged BG's can be given different time-interval boundaries.

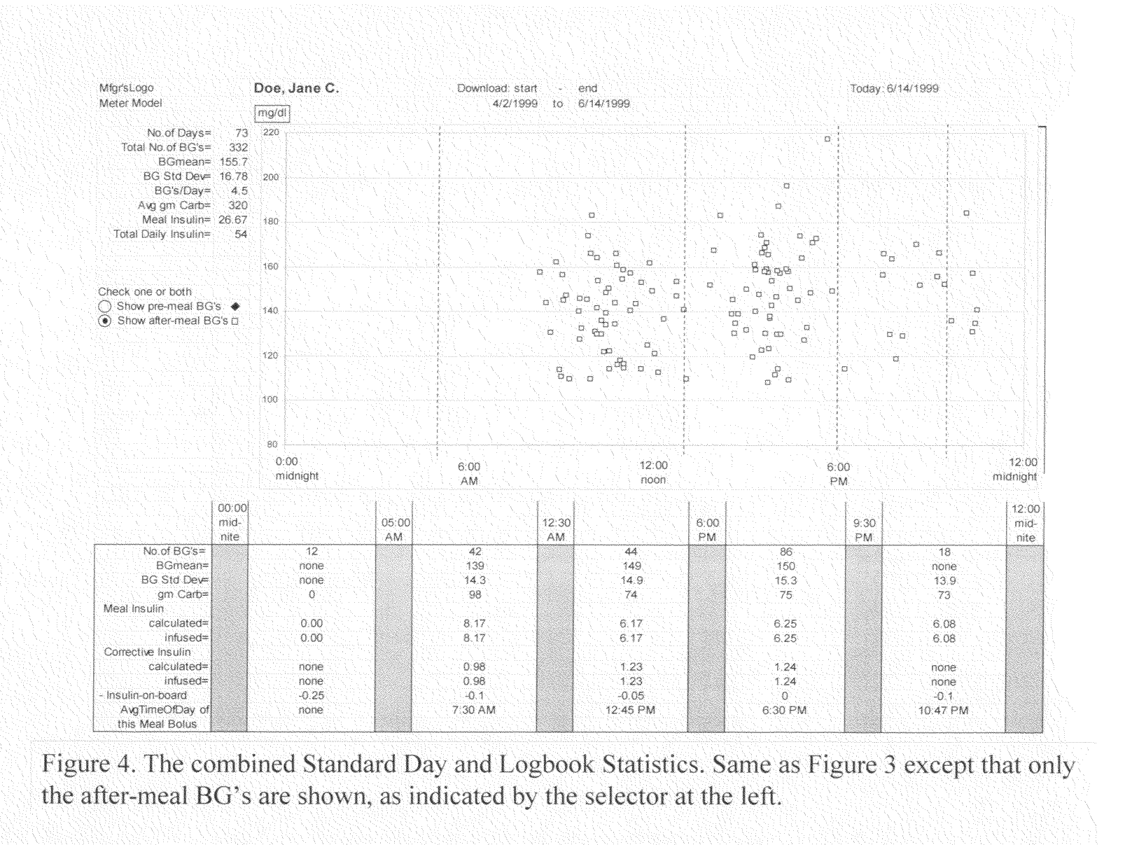

FIG. 5 illustrates an exemplary embodiment in which a single two-valued category parameter was used showing only pre-meal BG's. It shows a combined report form which uses expanding rectangles to group the data points into time intervals.

FIG. 6 illustrates an exemplary embodiment in which a single two-valued category parameter was used, showing only pre-meal BG's. It shows a report form in which freehand closured figures, drawn by the clinician with the mouse, are used to group the data points into time intervals.

FIG. 7 illustrates an exemplary embodiment in which a single two-valued category parameter was used, showing only pre-meal BG's. It shows a report form in which a subtraction feature is used to exclude data points from time intervals.

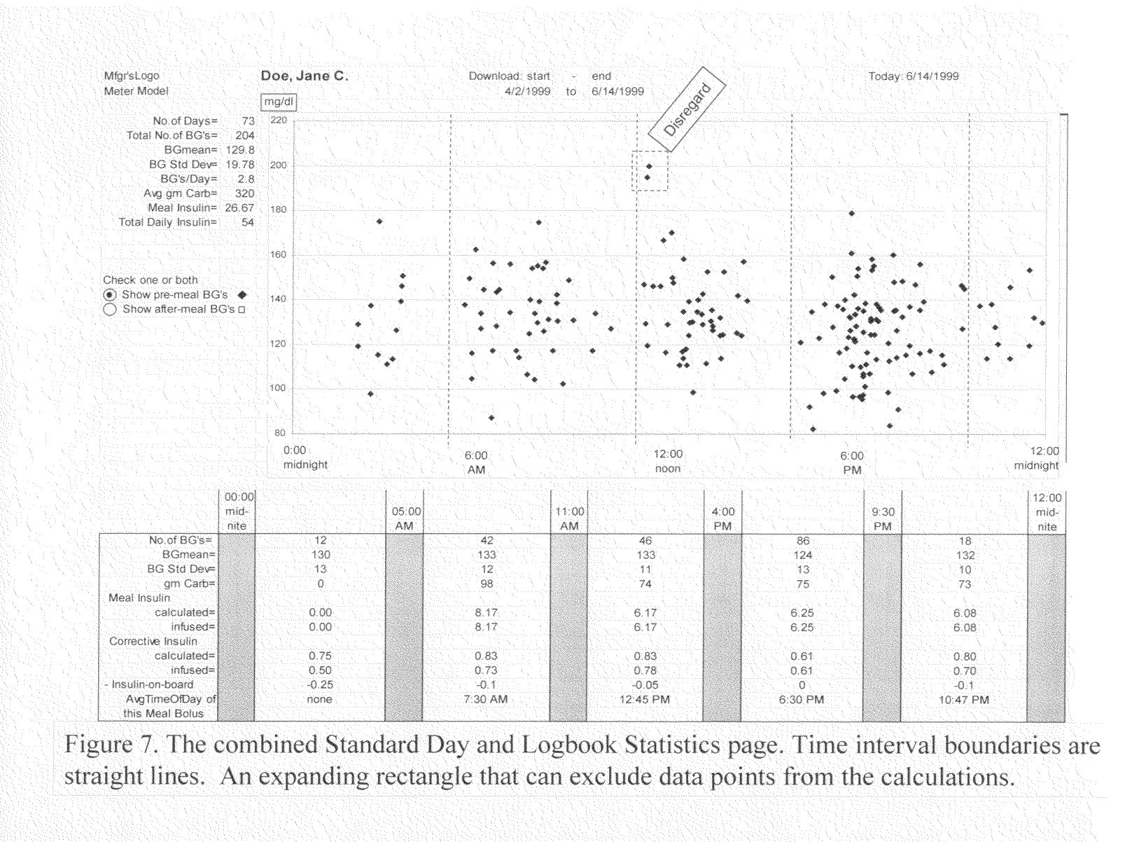

### DETAILED DESCRIPTION OF THE INVENTION

The present invention is a digital aid that will improve upon current diabetes care by making it possible for the physician or practitioner to analyze better and faster the downloaded data from the patient's insulin pump and/or blood glucose meter (portable devices). The downloaded data is comprised of the patient's recent history of the following parameters: Blood glucose (BG) test results, carbohydrate intakes, discrete Meal Insulin doses (Meal Boluses), discrete corrective insulin doses (Correction Boluses), Combined Boluses, and Remaining Insulin deductions from boluses. The discussion below focuses on BG data but may be applied to any of the parameters listed above. The invention provides a method enabling the patient at test-time to attach a category-label or flag to each BG test. In reading this it is important to know the distinction between a parameter, for instance BG with a value, for instance 120 mg/dl and a category variable, which carries the category that goes along with the parameter data item. The category variable in its simplest embodiment may have a value that is one of two categories (for example: binary or yes or no) whose meaning is known to the user. In more complex embodiments the category variable may have a text-string value for instance “pre-meal”. In still more complicated embodiments, a single parameter value may have more than one category field. The invention involves methods of assigning these categories to the parameters, for instance, the BG taken on Sep. 4, 2008 at 12:31 AM may have a value of 123 mg/dl and a category of “post-meal”. When the patient visits the clinician, the clinician can download the meter into a computer and analyze (statistically and graphically) the categories of BG's together, separately, or in various combinations. The download program also contains a feature that can digitally identify the meal times and the pre-meal and post-meal BG's (and other parameters) and assign categories to these parameters.

The invention is in four parts: 1) In the blood glucose meter, pump or other portable device with memory of blood glucose: a physical button or screen button on the portable device to enable the patient to assign a category to a BG. 2) In the computer-based downloading software: program features allowing faster graphical methods to be used to place the BG's into time-intervals during the day. These methods apply Graphical User Interface (mouse-based) methods upon the Standard Day scatter chart. 3) In the computer-based downloading software: a feature that can identify the meal times as being the time of the first meal bolus within a meal time interval. It can then assign a category to the pre-meal BG's that fall before the meal and can assign a category to the post-meal BG's identified as any BG's falling within a post-meal time window after the meal. 4) In the computer-based downloading software: program features allowing data with different categories to be displayed graphically and analyzed together, separately, or in different combinations.

### Part 1. In the Portable Device

### Portable Device, Simplest Embodiments:

One Two-valued Variable: In its simplest embodiment, the category is carried by a two-valued or binary variable with only two values: [the button was not pressed], or [the button was pressed]. This allows a choice between two categories whose union is the entire set of BG's, for instance [regular BG's] or [post-sugar-treatment BG's]. The meaning of the categories is not shown in the meter. It is remembered by the patient. The benefits from this simple two-valued system are numerous, despite the fact that only one choice can be made. Also the portable device can be kept simple and inexpensive.

Two Two-valued Variables: If there are two two-valued variables, then four combinations are possible, each of which can indicate a category whose meaning is known to the user. Two buttons or toggles on the LCD screen are available.

PORTABLE DEVICE, EMBODIMENTS OF MEDIUM COMPLEXITY: One Text-String Variable: By medium complexity is meant that the category variable (field) in the meter's memory is a text-string variable that offers a choice of a plurality of text-string values indicating category assignments. The possible categories are pre-chosen by the meter manufacturer and show in a multiple-choice list in the meter screen. Following the examples above, two of the choices would be, “Post-Meal” and “After-sugar-treated-low”. Portable devices having alpha-numeric input capability (like Palm or Rasberry devices) may incorporate an expanding list of categories that the user can add to, by keying into the box rather than selecting from the multiple choice list. The advantage of this embodiment over the simple two-valued embodiment is that BG's may be assigned one of several categories. The dis-advantage is that two categories cannot be assigned simultaneously to the same BG.

PORTABLE DEVICE, COMPLEX EMBODIMENTS: A very highly capable system involves the use of a plurality of category variables (fields); each of the variables, as in the mid-level embodiment, is a text-string and allows a plurality of category choices and a write-in capability. In such a system, each BG can be assigned to several categories without interfering with other assignments.

Part 2. In the Downloading Program: Alternate Methods for Grouping Blood Glucose Tests into Time-Intervals:

The computer-based downloading program uses the concept of dividing the day into time-intervals. The default names of the intervals are Breakfast, Lunch, Dinner, Bedtime, and Sleep, with typical starting and ending times. Both the names and the values may be changed. For returning patients, the values at the last office-visit are still active. The computer-based downloading software contains a tab that brings up an input page for inputting the patient's schedule of time intervals; this is the present state of the art. In addition, the invention enables the BG's to be grouped into time intervals graphically in the Standard Day scatter chart. The data dots are grouped and separated by manipulation of geometric figures on the Standard Day scatter chart using a mouse or other GUI (Graphical User Interface) means. The names of the time intervals appear above or adjacent to the geometric figures as well as in the Logbook at the heads of the columns. The names of the time intervals may be changed from their default values in either location by clicking on the present name. The new name may be selected from a drop-down list. The list contains the names Pre-Breakfast, Pre-Lunch, Pre-Dinner, Pre-Snack, Breakfast, Lunch, Dinner, Snack, Exercise, Post-Breakfast, Post-Lunch, Post-Dinner, Post-Exercise, Post-Snack, and Misc. If a name that is already assigned is selected, then the program will pool the data under the same name for statistics purposes. This feature may be used to create bedtime intervals that span midnight by adding two selections of data points to the bedtime interval. The geometric figures used to group the data points may be one of the following:

VERTICAL LINES: A set of vertical lines is the simplest method. Mouse-dragging them from side-to-side changes their location in time. The region between two lines is a time-interval. The invention enables the program to start with a small number of time-intervals (say, five) and then add more (up to, say, nine) if they are necessary or delete extras if there are too many by means of a pair of “add interval” and “delete interval” buttons. This feature may be enhanced by a marker that may be positioned by mouse to indicate the interval to be deleted or the location of a new interval to be added.

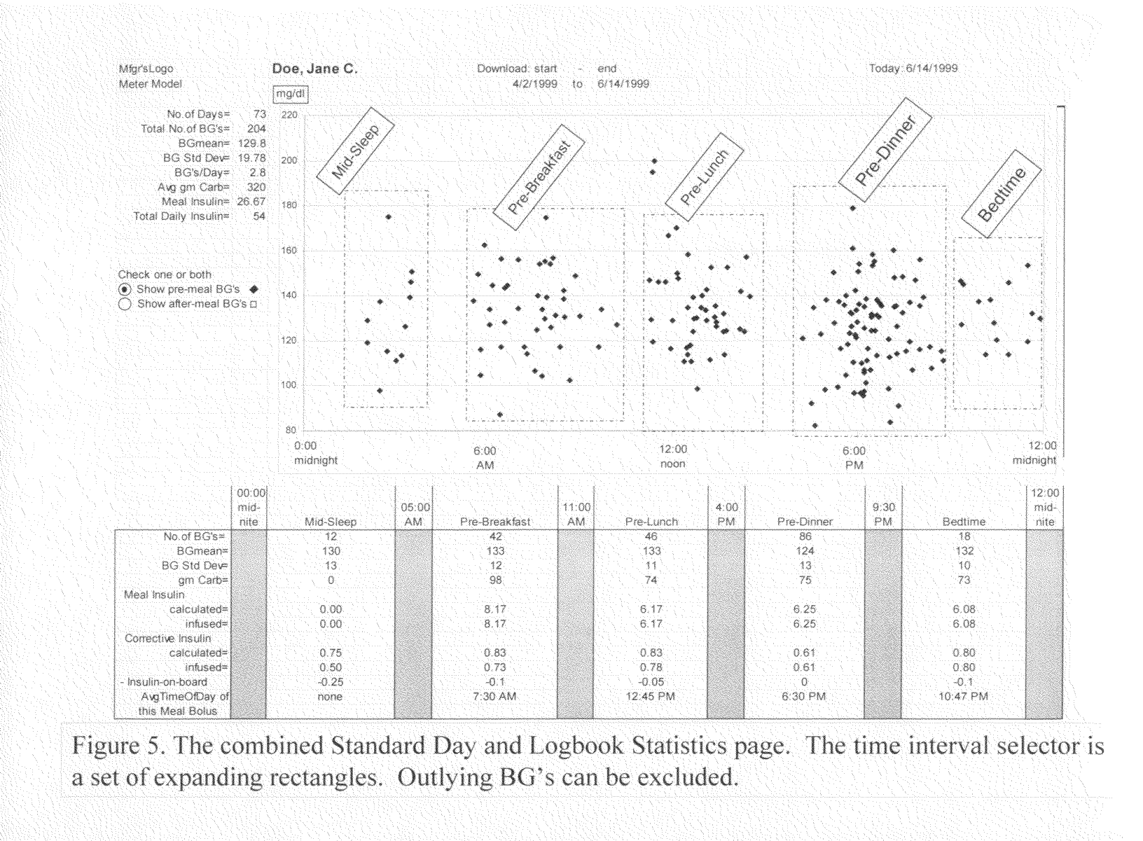

EXPANDABLE RECTANGLES: A set of stretchable and moveable rectangles like those in the Microsoft® AutoShapes feature may be used. These have the advantage that their height can be changed at will by mouse-stretching, making it possible to leave “outlier” BG's outside the rectangle if they are higher or lower than expected for a known reason. For instance a high BG following a sugar-treated low BG may be left outside the rectangle. (see FIG. 5).

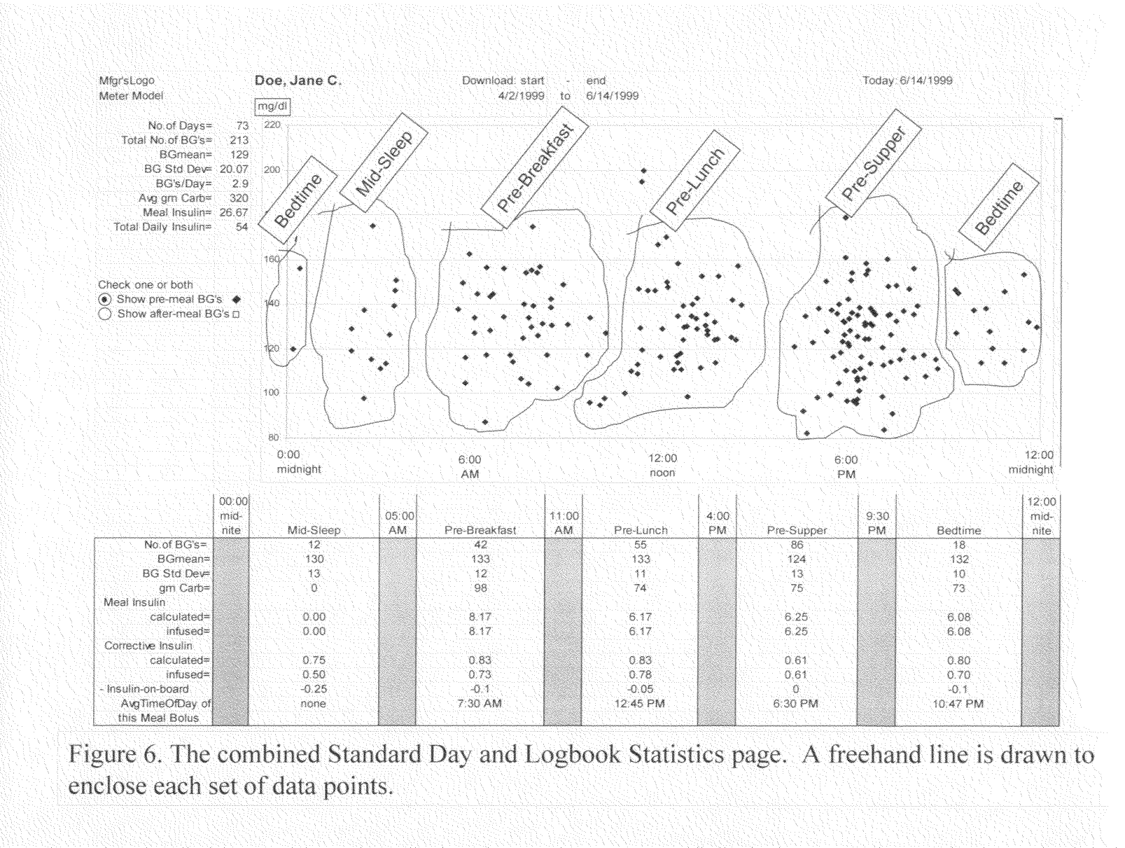

CLOSED FREEHAND FIGURES: Dots can be enclosed in a set of closed figures that are mouse-drawn in the manner of Microsoft® AutoShape “Scribble”. This allows the boundaries to be other than vertically straight on the sides, e.g. the data selected for the time-interval may be boot-shaped or anvil-shaped. The figure must be closed by making the line cross itself. (see FIG. 6)

### Part 3. In the Downloading Program; Digitally Assigning Categories

It is assumed that some patients will not be diligent about assigning categories in the portable device. This feature is called “automatic” in the program; it's purpose is to digitally flag or assign categories to BG's as an alternate system to assigning them in the portable device. For this feature to function, the time intervals must be set-up by the method of vertical lines. If that method is not selected, a reminder note pops up. The feature functions as follows: It identifies the meal time of each meal-containing interval of each day as being the time of the first meal bolus within a time interval and assigns the category of Post-Meal to any BG's falling within a post-meal time window, defined as follows: There is a post-meal time window for each meal-containing interval of each day. Their starting and ending times are in relation to the time of the meal in that interval and they are input in boxes labeled like the following example: Post-Meal time window from_hrs_min to_hrs_min after the meal. The default values are as follows, “ . . . from 1 hr 00 min to 3 hr 00 min after the meal” From one day to the next, the meal time for, say the breakfast interval may not be the same, so the location of, say the breakfast post-meal time window during the day may change even though location of the time window relative to the meal time does not. The post-meal time windows are not constrained by the boundaries of the main intervals; a post-meal time window may overlap into the next main time interval. In this case a BG may serve as a post-meal BG for one meal and a pre-meal BG for another interval, thus being counted twice. In an optional program feature, the ending times of the post-meal time windows are determined as the event that happens first: a) the time of the end of the post-meal window, or b) the end of the main interval. This avoids counting some BG's twice. These two options may be contained in the same embodiment, by use of a selector with two positions and labeled similar to the following example: Post-meal time windows overlap next time interval: [allowed] [not allowed]. There are also two options for applying the “pre-meal” category to pre-meal BG's and other pre-meal parameters. This category may be assigned to any and all BG's in the time interval but before the time of the meal, or only to those BG's occurring in a pre-meal time window. The pre-meal time windows are defined by a starting time in relation to the meal time; the ending time is always the time of the meal-bolus. The starting times are input in the same input page as the post-meal time windows like the following example: Pre-Meal time window starts at_hrs_min before meal.

If there are multiple meal boluses in a time interval, then the identification of post-meal time intervals is arranged in one of three ways: 1) The first meal bolus determines the one and only meal time and the single pair of time windows, the pre-meal and post-meal time window. 2) Each meal bolus has its own post-meal time window that defines its own group of post-meal BG's and other parameters. 3) The first meal bolus determines the first meal time and the start of the post-meal time window, and the last meal bolus determines the end of the post-meal time window.

### Part 4. In the Downloading Program; Analyzing Categorized Data

The BG data, including category assignments from the meter, are downloaded into a computer. In one of the screens is a digital selector for designating the method of category assignments. It has three positions:

a) Manual (categories were assigned in the meter in the manner outlined in Part 1)
b) Time Intervals (categories of pre-meal or post-meal and others are determined in the download program by location of BG's in the static time intervals in the manner of Part 2)
c) Automatic (categories of pre-meal or post-meal are determined in the manner of Part 3) using time windows that are located in time relative to the meal time and vary their time location from one day to the next.

In the downloading program the graphical feature shows the different categories or combinations of categories of BG's in different chart data series. The data series are represented by differently-shaped markers so that they show well in a black and white print copy. They also may be shown in different colors on the screen. For instance the pre-Breakfast BG's may be selected to show in the series represented by blue triangles. The Logbook Statistics page performs the statistics on all the data that are selected for viewing, i.e. if it is desired to perform statistics on only one of several data series, then only that data series should be selected for viewing. However, there is one exception; if the source of categories is selected to automatic, and if overlapping of post-meal windows and the next time interval is selected, then some BG's will fall into two categories, so they are displayed with the same marker style selected for the post-meal BG's, but to avoid multiple counting of these duplicated points in the statistics, the statistics page is locked-out, with a note recommending the use of the selection all BG's instead or allowing only one data series at a time for each use of the Statistics Page.

The statistics include the median BG of each interval, and enough information that all the parameters in equation (1) can be calculated. For instance, Insulin Remaining may be simply shown in the statistics page, or if bolus delivered is provided in the statistics page, and from elsewhere in the download the BG, the Correction Factor, the amount of carbohydrates, and the Carbohydrate-to-Insulin Ratio are available, then the Insulin Remaining can be calculated.

The Standard Day Scatter Chart may also be populated with other parameters than just BG's. Meal Boluses, amounts of carbohydrate, Correction Boluses, and Insulin Remaining may also be graphed. The same techniques are used as those used for BG's.

### Simplest Embodiments:

One Two-Valued Variable: In its simplest embodiment, the category is carried by a two-valued (or possibly binary) variable that can be activated by the patient to flag BG's in the portable device. Its two values are simply that the button in the meter was activated or not. The meaning represented by button-activation is known to the clinician and the patient. The choices for graphical and statistical analysis are: 1) all the data, 2) just the flagged data, and 3) just the not-flagged data. If a combination screen like those shown in the figures is used, then the choice can be made as shown in the figures, otherwise, the choice can be made in either a setup screen, the Logbook screen, or the Standard Day screen. Of these four locations the first two are preferred.

Two Two-Valued Variables: Four data series are allowed in the Standard Day Scatter Chart. On the computer screen, there is an actuator for each chart data series. Clicking on one of these will bring to the screen a selector box with two two-position toggles, on for each of the two two-valued variables. The choices on the toggles are [flagged] and [not flagged] or words to that effect. Four combinations are possible, corresponding to the four categories known to the patient. More than two two-valued variables may be incorporated in the same manner.

MID-LEVEL EMBODIMENTS: By mid-level complexity is meant that the category is carried in one text-string variable (field) that results in a collection of BG's, wherein each BG has one of several category assignments. In the downloading program, a selection of category is made for each data series. The selections include “(none)” which brings together a group of BG's to which no deliberate category assignment was made. The selector allows multiple selections, signifying that BG's assigned to any of the selected categories will be displayed in the said data series in the chart. A category is not allowed to be selected in more than one data series. Each BG is counted in the statistics only once. There are several possible embodiments for ways to select the categories for each chart series. One of these is described as follows:
There is a button for each data series. Upon clicking the button for a given data series, a drop-down list appears, listing the choices of categories, which are selected by highlighting. More than one category may be highlighted for a chart data series. Three data series are probably the most that can be viewed without confusion. To view more, different selections are made, and the page is re-printed.

COMPLEX EMBODIMENTS: there are a plurality of category variables; all are text-strings like that described for the mid-level embodiments. Each BG can have several category assignments, one for each category variable. For each chart data series, there is a drop-down list for selecting which categories to display in that chart data series. This selector shows the combined list of categories from all the category variables with the selections for that chart data series highlighted. (This method protects against the possibility that the patient may have assigned a certain category to different variables for different BG's or even for the same BG.) For each data series, one or more categories may be selected from the combined list. The selections include “(none)” or some other text-string indicating that the BG's were not assigned to a category by the patient. A category is not allowed to be selected in more than one data series. The Logbook Statistics report does not permit any BG to be counted statistically more than once, even if it is has more than one category assignment. There are several possible embodiments for ways to select the categories for each chart series. One of these is described as follows:
There is a button for each data series. Upon clicking the button for a given data series, a drop-down lists appears showing the combined list of categories. More than one category selection may be made. The selections are made by highlighting.

### REFERENCES

None

We claim:

1. A method for manually assigning digital categories to blood glucose tests (BG's) in a diabetic patient's portable blood glucose meter, insulin pump or other portable device by the patient at the time of testing by manual activation of a physical button or a selector on an LCD screen, wherein the categories may include but are not limited to pre-meal, post-meal, disregard, or other categories determined by the user, or wherein the button or selector has no label but its meaning may be understood by the patient and the practitioner, wherein the activation of the button or selector may allow one of two values or may allow a text string.

2. The method of claim 1 wherein all the methods described for BG's may also be used for meal boluses, correction boluses, and carbohydrate intakes.

3. (canceled)

4. The method of claim 1 wherein the category is a variable having two possible values, indicating whether the button or selector was pressed or not pressed.

5. The method of claim 4 wherein the category variable is binary.

6. The method of claim 1 wherein there is a plurality of category variables each having two possible values, indicating that the selector was activated or not activated.

7. The method of claim 1 wherein the category variable or plurality of category variables are short text strings.

8. The method of claim 7 wherein each category chosen for each variable is chosen from a fixed drop-down list in the LCD screen of the portable device.

9. The method of claim 7 wherein each category is chosen from a drop-down list in the LCD screen that can be added to through an alphanumeric input feature in the portable device.

10. The method of claim 9 wherein the portable device has alphanumeric input capability by virtue of being built into a palm device or a blackberry device.

11. (canceled)

12. (canceled)

13. An improvement the computer-based programs that download blood glucose meters, insulin pumps and other devices (downloading programs), for analyzing and graphically-displaying a plurality of differently-categorized data separately, all together, or together in combinations smaller than the total, wherein said combinations may employ differently-colored and/or differently-shaped markers for the BG's of different categories, and wherein mouse-based computer interface methods may be used to select BG's into time intervals.

14. The method of claim 13 wherein the category variables in the downloading program are character-strings.

15. The method of claim 14 wherein there are a plurality of chart data series, each of which is provided with a selector in which the selections are made for categories of a BG for inclusion in the said chart series.

16. The method of claim 15 wherein one of the category selections is “none” or some other phrasing indicating that no categories were assigned to those BG's by the patient.

17. The method of claim 15 wherein the selector for each chart data series is comprised of a drop-down list, showing the combined list of all the categories which have been assigned at least once in any of the category variables in the calendar period being studied.

18. The method of claim 15 wherein the selector allows multiple selections to be highlighted for the data series.

19. The method of claim 14 wherein the program prevents the selection of a category in more than one chart data series.

20. The method of claim 14 wherein there is only one text-string variable.

21. The method of claim 13 wherein the category variables in the downloading program are two-valued.

22. The method of claim 21 wherein the category variables are binary.

23. The method of claim 21 wherein there is a selector for each chart data series comprised of a rank of computer-screen interactive three-position selectors, one for each category variable, so that the desired combination of toggle positions for each chart series can be selected.

24. The method of claim 23 wherein the three selections are “flagged”, “not flagged”, or “n/a” or labels to that effect that BG's will be shown if the parameter is a) flagged or b) not flagged or c) that the BG will not be shown in said data series unless other parameters so dictate.

25. The method of claim 24 wherein if all the parameter selectors show the value indicative of c) in claim 24, then the said chart series will not show in the Scatter Chart.

26. The method of claim 23 wherein a given combination of variable values is allowed in only one data series.

27. The method of claim 21 wherein there is only one two-valued category variable.

28. The method of claim 27 wherein there are only two data series, each pre-assigned to one of the two conditions of the single two-valued category variable and wherein the selector on the computer screen is similar to that shown in the Figures.

29. (canceled)

30. (canceled)

31. The method of claim 13 wherein the order of superposition of chart data series in the chart can be changed.

32. The method of claim 17 wherein pre-existing methods are adopted for changing the order of superposition of the data series.

33. (canceled)

34. The method of claim 13. for summing, averaging and statistically analyzing a plurality of differently-categorized data separately or together.

35. The method of claim 34 wherein the data are blood glucose test results

36. The method of claim 34 wherein the data used in the statistical computations are all the data appearing on the Standard Day scatter chart.

37. The method of claim 34 wherein the data used in the statistical computations are all the data appearing on the Standard Day scatter chart, except that the program counts each BG only once in the statistical calculations.

38. The method of claim 13 wherein discrete time-indexed medical data are grouped by graphical means into groups representing time-intervals in a scatter chart of points representing the discrete test results as a function of time in which all the data for a calendar period are plotted in the same standard 24 hour day (Standard Day scatter chart)

39. The method of claim 38 wherein the discrete medical data are blood glucose tests.

40. The method of claim 38 wherein the discrete medical data are amounts of carbohydrates consumed.

41. The method of claim 38 wherein the discrete medical data are amounts of medicine administered.

42. The method of claim 41 wherein the amounts of medicine are discrete insulin doses (boluses).

43. The method of claim 38 wherein the graphical means are operations of a mouse, touchpad, trackball or other device common to Graphical User Interface (GUI) technology, wherein such methods are used upon the Standard Day scatter chart.

44. The method of claim 43 wherein the discrete medical data are grouped into groups representing time-intervals in the Standard Day scatter chart by mouse-dragging-and-dropping of vertical lines representing the time-boundaries between these time-intervals.

45. The method of claim 44 wherein the number of time intervals may be increased or decreased from a default value.

46. The method of claim 45 wherein the number of intervals is changed one interval at a time by a pair of buttons, one to add an interval and one to delete an interval.

47. The method of claim 46 wherein a new interval will be added at the location of the mouse or a cursor or marker, and wherein an unwanted interval will be deleted at the location of the mouse or cursor or marker will be deleted.

48. The method of claim 47 wherein a marker may be positioned by mouse to select an interval for deletion or to indicate which side of the selected interval will be the location of a new interval to be added.

49. The method of claim 48 wherein the position of the marker relative to the centerline of the selected interval determines which side of the selected interval the new interval will be placed on.

50. The method of claim 43 wherein the discrete medical data are grouped into groups representing time intervals by enclosing them in stretchable closed geometric figures that are not required to enclose all the data on the test-result axis (Y axis) between the maximum and minimum times.

51. The method of claim 50 wherein the stretchable closed geometric figures are rectangles.

52. The method of claim 50 wherein the stretchable closed geometric figures are drawn freehand by use of the mouse or mouse-substitute, wherein said figures are closed by drawing the line to cross itself and wherein the closed figures are not required to enclose all the data points between their maximum and minimum times.

53. The method of claim 50 wherein additional statistics are shown that are calculated for the whole day from only those BG's selected into time intervals.

54. The method of claim 49 wherein the time intervals are named in the Standard Day scatter chart at the top, above the time-intervals (in addition to the names traditionally appearing in the Logbook above the appropriate columns.).

55. The method of claim 49 wherein the names for the time-intervals are written within the enclosed selection of data points or on an adjacently-placed tag as shown in FIG. 6 (in addition to the names traditionally appearing in the Logbook above the appropriate columns.).

56. The method of claim 43 wherein the names can be changed in the Standard Day scatter chart by clicking on the default name (or previously-assigned name) and changing it.

57. The method of claim 43 wherein the names can be changed in the Logbook by clicking on the default name (or previously-assigned name) and changing it.

58. The method of claim 56 or claim 57 wherein clicking on the time-interval name causes a dropdown list to appear containing a permanent list of commonly-used names, and wherein either said dropdown list or direct typing may be used to change the time-interval name to a name that may be on the list or not.

59. The method of claim 43 wherein two data selections may be given the same time-interval name, and if they do, the data will be combined, for instance a bedtime interval with data before and after midnight.

60. The method of claim 58 wherein clicking on the name of a time-interval will cause a drop-down box to appear showing a list of common names for time-intervals and allowing new input, and wherein words show above or before the name-change box saying, “create or add to” or words to the effect that selection of a time-interval name already in use will add to the data in the said time interval.

61. The method of claim 59 wherein a subtraction option is also available.

62. An improvement for computer-based programs that download the portable devices (downloading programs) wherein digital categories of pre-meal and post-meal are digitally are assigned to BG's within pre-meal and post-meal time windows which are individually determined for every meal-containing interval of every day, wherein each meal time is determined as the time of the first meal bolus in a time interval and wherein a post-meal time window is located in time relative to each day's meal time for the interval and extends from a delay-time after the meal to an ending time after the meal, and wherein post-meal BG's, carbohydrate intakes, Meal Boluses, Correction Boluses, and Insulin Remaining amounts are identified as post-meal if they fall inside the post-meal time window or identified as pre-meal if they fall inside a pre-meal time window.

63. The method of claim 62 wherein, in each day, in each time interval containing a meal, pre-meal and post-meal BG's are identified within pre-meal and post-meal time windows which are individually determined for every meal-containing interval of every day

64. The method of claim 63 wherein each meal time is determined as the time of the first meal bolus in a time interval and wherein a post-meal time window is located in time relative to each meal time (of a single interval of a single day), for the interval and extends from a delay-time after the meal to an ending time after the meal, and wherein post-meal BG's are identified as any BG's falling inside the post-meal time window.

65. The method of claim 63 wherein the meal times are determined as the time of the first meal bolus in a time interval, and wherein the pre-meal and post-meal time windows are determined relative to the meal time, for each meal-containing time interval of each day.

66. The method of claim 65 wherein a post-meal window of time is determined as starting and ending at times relative to the meal time, that is, a post-meal window starts after a delay-time after the meal and ends at a relative end time after the meal.

67. The method of claim 65 wherein a pre-meal time window extends from a relative starting time before the meal to meal time and wherein any BG falling inside a pre-meal time window is identified as a pre-meal BG.

68. The method of claim 65 wherein a pre-meal time window extends from the start of the main interval to meal time and wherein any BG falling inside this pre-meal time window is identified as a pre-meal BG.

69. The method of claim 63 wherein the starting delays and relative end time of a post-meal time window and the starting time of a pre-meal window for a time interval in the standard day for continuing daily use are input individually for each time interval that has a meal, wherein the input value replaces a default value wherein these values remain in the memory and are used for the said interval on every day until changed.

70. The method of claim 65 wherein the Pre-Meal BG's are identified as all BG's occurring within a Pre-Meal window of time that starts at a relative start time before the meal, and ends at meal time.

71. The method of claim 63 wherein the pre-meal relative start times are input individually for the applicable intervals.

72. The method of claim 65 wherein all the BG's occurring in an post-meal time window are assigned the category of post-meal, and all the BG's occurring in a pre-meal time window are assigned the category of pre-meal.
